SEQUENCE LISTING (19 sequences)

SEQ ID NO: 1 - protein, 467 aa, synthetic construct
MGWSCIILFLVATATGVHSQVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 233 aa, synthetic construct
MGWSCIILFLVATATGVHSDIVMTQSPATLSVTPGDRVTLSCRASQSISDYLYWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 585 aa, synthetic construct
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 4 - DNA, 1401 bp, synthetic construct
  source 1..1401: mol_type = other DNA
atgggttggagttgcatcatcctatttctagtggccaccgctaccggcgtgcactcccaagtgcaactacaacaaagtggtgcagaactcgtcaaacccggagcttccgtgaagctgtcctgcaaggcctctggctacaccttcaccaactacgacatcaactgggtgcggcagcggcctgagcaaggcctggaatggatcggctggatcttccctggcgatggctccacccagtacaacgagaagttcaagggcaaggctacactgaccaccgacaccagcagctctaccgcctacatgcagctgtccagactgacatccgaggactccgccgtgtacttttgtgccagacagaccacagctacctggttcgcctattggggccagggcaccctggtgaccgtgtctgctgcctccaccaagggcccttctgtgtttcctctggctccttcttccaagtccacctctggcggaacagccgctctgggctgtctggtcaaggactactttccagagccagtgacagtgtcttggaactccggcgctctgaccagcggcgtgcataccttccctgccgtgctgcagtccagcggactgtacagcctgtccagcgtggtcacagtgccttcctcctccctgggaactcagacgtacatctgcaacgtgaaccacaagccttctaacaccaaagtggataagaaggtggaacctaagtcctgcgacaaaacccacacctgtcctccctgccctgctccagagctgctgggcggcccttcggtgttcctgttccctcctaagcccaaggataccctgatgatctcccggacccctgaggtgacctgcgtggtggtggacgtgtcccacgagaatcctgaagtgaaattcaactggtacgtggatggcgtggaagtgcataacgccaagacaaagcctagagaggaacagtacaacagtacctacagagtggtgtccgtgctgaccgtgctgcaccaggactggctgaacggcaaagagtacaagtgcaaggtctctaacaaggctctgcctgcccccatcgagaagacaatcagcaaggccaaaggacagccccgggaacctcaagtttataccctgcctccctctcgggaagagatgaccaagaaccaggtgtccctgtggtgcctggtgaaaggcttctaccctagcgatatcgccgtcgagtgggagtccaacggccagcctgagaacaactacaagaccaccccacctgtcctggactccgacggctcctttttcctgtactccaagctgaccgtggacaagtccagatggcagcagggcaatgtgttctcctgttctgtgatgcacgaggccctgcacaaccactacacccagaagtctctgtctttatctcccggcaag

SEQ ID NO: 5 - DNA, 699 bp, synthetic construct
  source 1..699: mol_type = other DNA
atgggttggagttgcatcatcctatttctagtggccaccgctaccggcgtgcactccgatatcgtgatgactcaaagtccagcaactctgtctgtcacacctggcgacagagtgaccctcagctgtagagccagccaatctatctccgactacctgtactggtatcagcagaagtcccacgagtccccacggctgctgatcaagtacgcctctcagtccatctctggcatccccagccggttctccggctccggctctggatctgatttcaccctgtccatcaactccgtggaacctgaggacgtgggcgtgtactactgccagaacggccactcctttcctctgaccttcggcgctggcaccaagctggagctgaaacggaccgtggctgctccttctgtgtttatcttccctccttccgacgaacagctgaaatccggaaccgccagcgtggtgtgcctgctgaacaacttctaccccagagaggccaaggtgcagtggaaggtggacaacgccctgcagtctggcaattctcaagagtccgtcaccgagcaggactccaaggactctacctacagcctgtcctccaccctgaccctctctaaggccgattacgagaagcacaaggtctatgcctgcgaagttacacatcagggcctgagctctcctgtgactaaatccttcaacagaggcgagtgt

SEQ ID NO: 6 - DNA, 1761 bp, synthetic construct
  source 1..1761: mol_type = other DNA
atgcatagtagtgcactactatgctgcctagtgctgctgaccggcgtgcgggcttctcctggacaaggcacccagtccgagaactcttgcacccattttcctggcaacctgcctaacatgctgcgggatctgcgggacgccttttcccgggtgaagaccttttttcagatgaaagatcagctggacaacctgctgctgaaggaatccctgctggaagacttcaagggctacctgggctgtcaggctctgagcgagatgatccagttctatctggaagaagtgatgcctcaggccgagaaccaggatcctgatatcaaggctcatgtgaactccctgggcgagaacctgaaaaccctgcgcctgagactgagaagatgccacagattcctgccttgcgagaacaagtccaaggccgtggaacaggtcaaaaacgccttcaacaagctgcaggagaagggcatctacaaggctatgtccgagttcgacatcttcatcaactatatcgaggcttacatgaccatgaagatcagaaacagctccggcggaggcggttctggcggcggcggctccggcggcggaggatcctctccaggacagggcacccagtctgaaaactcctgcacccacttccccggcaacctgcccaacatgctgagagacctgagggatgccttcagccgggtgaagaccttcttccagatgaaggaccagctggataacctgcttctgaaggagtccctgctcgaagacttcaagggctacctgggctgccaggccctgtctgaaatgatccagttctacctggaagaggtgatgccccaggctgagaaccaggaccccgatatcaaggcccacgtgaactctctgggcgagaatctgaagacactgcggctaagactgcggagatgtcaccggttcctgccatgcgagaacaagtccaaagctgttgagcaggtgaaaaacgctttcaacaagctgcaagagaagggcatctacaaggccatgtctgagttcgacatcttcatcaactacatcgaggcctacatgaccatgaagatccggaacggatctggcggcggcgacaagacacacacctgtcccccttgtcctgcccctgagctgctcggcggaccttctgtgtttctgttccctcctaagcctaaggacaccctgatgatttccagaacacccgaagtgacctgcgtggtggttgacgtgtctcacgagaaccctgaggtcaagttcaactggtacgtggacggagtggaagtgcacaatgccaagaccaaaccccgggaagagcaatacaactccacctacagagtggtgtccgtgctgaccgtgctgcaccaggactggctgaacggcaaggagtacaagtgcaaggtgtccaacaaagccctgcctgctcctatcgagaagaccatcagcaaggccaagggccagcctagagagcctcaagtgtacaccctgcctccatccagagaggaaatgacaaagaatcaggtctcgctcacctgcctggtgaaaggcttctacccttccgacatcgccgtcgaatgggagtctaatggccagcccgagaataactacaagaccacccctcctgtgctggactctgacggctccttcttcctggcctccaagctgacagtggacaagagcagatggcagcagggcaacgtgttctcctgctccgtgatgcacgaggcactgcacaaccactacacccagaagagcctgtctctgtctcctggcaag

SEQ ID NO: 7 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 8 - protein, 330 aa, synthetic construct [sequence omitted]

SEQ ID NO: 9 - protein, 107 aa, synthetic construct [sequence omitted]

SEQ ID NO: 10 - protein, 448 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1344 bp, synthetic construct
  source 1..1344: mol_type = other DNA
caggttcagctgcaacagtctggcgcagaactggtaaaaccaggtgcatccgtgaaactgagctgcaaggcatctggctacacttttaccaattacgacattaactgggtacgccagcgtccggaacagggtctggaatggattggctggatcttcccgggcgacggttccactcaatataacgagaaatttaaaggtaaagcgactctgactactgatacgtcctcctctaccgcttacatgcagctgtcccgtctgacctctgaggattctgctgtctacttctgtgcccgtcagaccaccgctacttggttcgcctattggggccagggtactctggttactgtttctgcagcatctacgaaaggtccgtctgtatttccgctggcaccgagcagcaagtccacttctggtggtactgctgcactgggctgcctggtgaaagattatttcccggaaccggtgaccgtatcttggaactctggtgcactgacctctggtgtgcacacgttcccagcagttctgcagtcttctggcctgtatagcctgtcctctgttgtgaccgttccttcttcctctctgggcacgcagacctacatctgcaacgtaaaccacaaaccgtccaacaccaaagtcgacaaaaaagtggaaccgaaatcctgtgataaaacccacacttgcccgccgtgtccagcacctgaactgctgggtggtccgagcgtattcctgtttccgccgaaaccgaaagacaccctgatgatttctcgtactccggaagtgacctgcgtagtcgtagacgttagccacgaaaacccggaagtgaaattcaactggtacgtcgatggcgttgaagtgcacaacgcgaaaaccaagccgcgcgaagaacagtacaactccacctatcgtgtcgtatccgtactgactgtgctgcaccaggactggctgaacggtaaagaatacaaatgcaaagtttctaacaaagctctgccggcgccgatcgaaaaaaccatttccaaagccaaaggccagccacgtgaaccacaggtttacaccctgccgccgagccgtgaagagatgaccaagaaccaagttagcctgacttgcctggtgaaaggtttttacccgtccgacattgcggtagaatgggaatccaatggtcaaccggagaacaactacaaaaccactccgccagttctggactctgatggttctttcttcctgtattccaaactgactgttgataaaagccgttggcagcagggcaacgttttctcctgctccgttatgcacgaagcactgcacaaccattacacccagaaatccctgtctctgtctccgggcaag

SEQ ID NO: 12 - protein, 118 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSA

SEQ ID NO: 13 - protein, 214 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 5 aa, synthetic construct
NYDIN

SEQ ID NO: 15 - protein, 17 aa, synthetic construct
WIFPGDGSTQYNEKFKG

SEQ ID NO: 16 - protein, 9 aa, synthetic construct
QTTATWFAY

SEQ ID NO: 17 - protein, 11 aa, synthetic construct
RASQSISDYLH

SEQ ID NO: 18 - protein, 7 aa, synthetic construct
YASQSIS

SEQ ID NO: 19 - protein, 8 aa, synthetic construct
QNGHSFPL

(19) United States
(12) Patent Application Publication
(10) Pub. No.: US 2024/0392007 A1
(43) Pub. Date: Nov. 28, 2024

(54) HETERODIMERIC PROTEIN AND APPLICATION THEREOF

(71) Applicant: SHENGHE (CHINA) BIOPHARMACEUTICAL CO., LTD., Nanjing, Jiangsu (CN)

(72) Inventors: Chong ZHOU, Nanjing (CN); Xiaoling JIANG, Nanjing (CN); Liusong YIN, Nanjing (CN)

(73) Assignee: SHENGHE (CHINA) BIOPHARMACEUTICAL CO., LTD., Nanjing, Jiangsu (CN)

(21) Appl. No.: 18/694,554

(22) PCT Filed: Sep. 23, 2022
(86) PCT No.: PCT/CN2022/120730

§ 371 Date: Mar. 22, 2024

(30) Foreign Application Priority Data
Sep. 27, 2021 (CN) 202111135075.7

(51) Int. Cl. C07K 16/28; A61K 39/00; A61P 35/00; C07K 16/24

(52) U.S. Cl. CPC C07K 16/2827; A61P 35/00; C07K 16/244; A61K 2039/505; C07K 2317/31; C07K 2317/565

(57) ABSTRACT

In the field of biomedicine, a heterodimeric protein and application thereof. The heterodimeric protein includes: (1) a light chain and a first heavy chain, where the light chain and the first heavy chain are combined to form a targeting moiety that exhibits binding specificity to a tumor antigen or an immune checkpoint, and the tumor antigen or the immune checkpoint includes B7H3; (2) a second heavy chain, wherein the second heavy chain includes a Fc region and an immunomodulator fused to the Fc region, and the immune regulator includes IL-10. The results of affinity test showed that the heterodimeric protein had high affinity for B7H3 and IL-10 receptors. The results of pharmacodynamic experiments in vivo showed that the heterodimeric protein had good anti-tumor activity.

### TECHNICAL FIELD

The present invention belongs to the technical field of biomedicine, and relates to a heterodimeric protein and application thereof.

### BACKGROUND

B7H3 (CD276) was identified as a member of the B7 family, and TLT-2 was identified as its potential receptor in 2001. Recent studies have shown that B7H3 is both an immune co-stimulatory and co-inhibitory molecule, and has anti-tumor activity and can trigger immune escape. Therefore, its tumor-associated antigen properties are mostly used in antibody drug development to kill tumor cells with high expression of B7H3 through ADC drugs, ADCC mechanisms, etc.

Northern blot analysis showed that B7H3 mRNA is widely expressed in a variety of normal tissues including liver, small intestine, pancreas, testis, heart, colon, etc, but not expressed in human peripheral blood leukocytes. However, B7H3 protein is only expressed at low levels, but its expression can be induced in B cells, T cells, monocytes or NK cells by granulocyte-macrophage colony-stimulating factor (GM-CSF) or lipopolysaccharide (LPS) stimulation.

Soluble B7H3 (sB7H3) has been shown to be released by monocytes, dendritic cells (DCs) and activated T cells. High expression of B7H3 is negatively correlated with tumor size in breast cancer patients. B7H3 was expressed in 93% of ovarian tumors compared with normal ovarian tissue, and B7H3 expression is associated with staging, high recurrence and low survival rates in ovarian tumor. B7H3 expression is also significantly elevated in colorectal cancer and may be involved in the development of colorectal cancer. In addition, B7H3 protein is also expressed in prostate cancer, pancreatic cancer, squamous cell carcinoma (SCC), non-small cell lung cancer (NSCLC) and gastric cancer. The aberrant expression of B7H3 in many tumors suggests that B7H3 may be a useful marker for the study of tumorigenesis, progression, diagnosis and treatment.

Therefore, we can target the physiological functions of B7H3 in tumor growth, migration and invasion to develop antibody drugs that use ADC drugs, ADCC mechanism, etc. to kill tumor cells that express high levels of B7H3 in the tumor microenvironment.

IL-10 is mainly secreted by activated T cells and antigen-presenting cells, and IL-10 receptor (IL-10R) expression is upregulated in CD8+ T cells during antigen recognition. IL-10 mediates multiple activities by a specific cell surface receptor complexes. The IL-10 receptor contains two distinct chains, IL-10R1 and IL-10R2, both of which belong to the class II cytokine receptor family (CRF2). IL10 reduces the inflammatory response when bacterial infections and tissue injury, inhibits the inflammatory response induced by T cells (Th17) and macrophages (IL-12/23) and reduces the tumor-associated inflammatory responses. IL-10 can antigen-activate the proliferation and toxicity of CD8+ T cells at high concentrations.

Anti-tumor mechanisms of the IL-10 are: a. it can activate activity and expansion of CD8+ T cells inside the tumor; b. it can increase activity and expansion of antigen-specific T lymphocytes inside the tumor; c. it has a memory function for the rejection of tumor, the in vivo assays for drug testing show that after the tumor disappeared from the IL-10 drug, the tumor cells doesn't grow in the mice when given again for inoculation. The main reason is that IL-10 can enhance the survival of antigen-specific CD8+ T cells and act as a tumor vaccine. Clinical trials have also demonstrated that IL-10 can increase the number of PDL1-specific CD8+ positive cells inside the tumor, producing a long-lasting anti-tumor effect when used in combination with PDL1 antibodies. However, there are currently no marketed drugs targeting IL-10.

IL-10 promotes expansion and survival of CD8+ T cells that target specific antigens, which are positively correlated with tumor killing by immune cells. Although several studies have shown that immunomodulators can be used to exert anti-tumor effects in animal models and in cancer patients, however, their use is greatly limited by the short half-life and systemic toxicity associated with the use thereof. CN200880117225.8 describes a chimeric construct containing interferons linked to the C-terminus of an antibody directed against a tumor-associated antigen. However, the fusion proteins expressed by such chimeric constructs are usually very unstable in vivo. Furthermore, their expression yields are usually too low to be used for large-scale industrial production.

### SUMMARY

The purpose of the present invention is to provide a heterodimeric protein and use thereof. The heterodimeric protein has high affinity to both B7H3 and IL10 receptors, and has good anti-tumor activity.

The technical schemes of the present invention are as follows.

A heterodimeric protein and use thereof, comprising:
(1) a light chain and a first heavy chain, wherein the light chain and the first heavy chain are combined to form a targeting moiety that exhibits binding specificity to a tumor antigen or an immune checkpoint; and
(2) a second heavy chain, wherein the second heavy chain comprises a Fc region and an immunomodulator fused to the Fc region;
and the light chain, the first heavy chain, and the second heavy chain are combined to form the heterodimeric protein.

Further, the tumor antigen or the immune checkpoint is selected from one or more of B7H3, B7H4, B7H5, BTLA, CD27, CD28, CD153, CD40, CD40L, CD70, CD80, CD86, CD96, CD112, CD134, CD137, CD137L, CD152/CTLA-4, CD155, CD223, CD226, CD252/OX40L, CD258, CD273/PD-L2, CD274/PD-L1, CD278, CD279, CD357, DR3, Galectin-9, GITRL, HVEM, ICOSL/B7RP1/B7H2, IDO, TIGIT, TIM-3, TLIA, MART-1/MelanA, gp100, tyrosinase, TRP-1, TRP-2, MAGE-1, MAGE-3, BAGE, GAGE-1, GAGE-2, p15, CEA, p53, Ras, HER-2/neu, BCR-ABL, E2A-PRL, H4-RET, IGH-IGK, MYL-RAR, Epstein Barr virus antigen EBVA, human papillomavirus antigen E6 or E7, TSP-180, MAGE-4, MAGE-5, MAGE-6, RAGE, NY-ESO, erbB, p185erbB2, p180erbB-3, c-met, nm-23H1, PSA, TAG-72, CA 19-9, CA 72-4, CAM 17.1, NuMa, K-ras, β-linked protein, CDK4, Mum-1, p 15, p 16, 43-9F, 5T4, 791Tgp72, methotrexate, β-HCG, BCA225, BTAA, CA 125 CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, MUC16, IL13Rα2, FRα, VEGFR2, Lewis Y, FAP, EphA2, CEACAM5, EGFR, CA6, CA9, GPNMB, EGP1, FOLR1, endothelial receptor, STEAP1, SLC44A4, binding element-4, AGS-16, guanylyl cyclase C, MUC-1, CFC1B, integral protein α3 chain, TPS, CD19, CD20, CD22, CD30, CD72, CD180, CD171, CD123, CD133, CD138 CD37, CD70, CD79a, CD79b, CD56, CD74, CD166, CD71, CLL-1/CLEC12A, ROR1, phosphatidylinositol proteoglycan 3, mesothelin, CD33/IL3Ra, c-Met, PSCA, PSMA, glycolipid F77, EGFRVIII, BCMA, GD-2, MY-ESO-1 and MAGE A3.

Furthermore, the tumor antigen or the immune checkpoint is B7H3.

Further, the light chain contains a complementary determining region (CDR), and the complementary determining region comprises an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to the amino acid sequence of the corresponding CDR of the light chain of an antibody specifically binding to a tumor antigen or an immune checkpoint.

Furthermore, the light chain of the antibody specifically binding to the tumor antigen or the immune checkpoint contains LCDR1 of the amino acid sequence shown by SEQ ID NO: 17, LCDR2 of the amino acid sequence shown by SEQ ID NO:18, and LCDR3 of the amino acid sequence shown by SEQ ID NO:19.

Further, the light chain comprises a variable region, and the variable region comprises an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence comprised in the variable region of the light chain of an antibody specifically directed against a tumor antigen or an immune checkpoint.

Furthermore, the amino acid sequence of the variable region of the light chain is shown by SEQ ID NO:13, or is an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO:13.

Further, the amino acid sequence of the light chain is shown by SEQ ID NO:2, or is an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO:2.

Furthermore, the nucleotide sequence encoding the light chain is shown by SEQ ID NO: 5, or is a nucleotide sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO:5.

Further, the first heavy chain comprises a complementary determining region (CDR), and the complementary determining region comprises an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to the amino acid sequence of the corresponding CDR of the light chain of an antibody specifically binding a tumor antigen or an immune checkpoint.

Furthermore, the first heavy chain of the antibody specifically binding to the tumor antigen or the immune checkpoint contains HCDR1 of the amino acid sequence shown by SEQ ID NO:14, HCDR2 of the amino acid sequence shown by SEQ ID NO:15, and HCDR3 of the amino acid sequence shown by SEQ ID NO:16.

Further, the first heavy chain comprises a variable region, and the variable region comprises an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence comprised in the variable region of the light chain of an antibody specifically directed against a tumor antigen or an immune checkpoint.

Furthermore, the amino acid sequence of the variable region of the first heavy chain is shown by SEQ ID NO:12, or is an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO: 12.

Further, the amino acid sequence of the first heavy chain is shown by SEQ ID NO: 1, or is an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO:1.

Furthermore, the nucleotide sequence encoding the first heavy chain is shown by SEQ ID NO:4, or is a nucleotide sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO:4.

Further, the second heavy chain comprises one or more residues to enable the heterodimerization of (1) and (2) as described above by covalent bond.

Further, the immunomodulator is linked to the Fc region of the antibody that specifically binds the tumor antigen or the immune checkpoint.

Furthermore, the second heavy chain comprises a constant region of an immunoglobulin selected from IgG1, IgG2, IgG3 and IgG4.

Further, the second heavy chain comprises one or more Fc regions of the same or different types, and the Fc regions are fused to the immunomodulator by means of a polypeptide linker.

Furthermore, the polypeptide linker is a 5-30 amino acid.

Furthermore, the polypeptide linker is (GGGGS) n, wherein n=1-6.

Further, the second heavy chain comprises two or more immunomodulators of the same or different types, and the two or more immunomodulators are fused to each other and to the Fc region.

Further, the immunomodulator enhances the immune response.

Further, the immunomodulator reduces the immune response.

Further, the immunomodulator is a cytokine, cytokine receptor, growth factor, hormone or extracellular matrix molecule.

Furthermore, the immunomodulator is selected from one or more of IL-1, IL-2, IL-2 Rα, IL-2 Rβ, IL-3, IL-3 Rα, IL-4, IL-4 Rα, IL-5, IL-5 Rα, IL-6, IL-6 Rα, IL-7, IL-7 Rα, IL-8, IL-9, IL-9 Rα, IL-10, IL-10R1, IL-10R2, IL-11, IL-11 Rα, IL-12, IL-12 Rα, IL-12 Rβ2, IL-12 Rβ1, IL-13, IL-13 Rα, IL-13 Rα2, IL-14, IL-15, IL-15Rα sushi, IL-16, IL-17, IL-18, IL-19, IL-20, IL-20R1, IL-20R2, IL-21, IL-21 Rα, IL-22, IL-23, IL-23R, IL-27 R, IL-31 R, G-CSF-R, LIF-R, OSM-R, GM-CSF-R, Rβc, Rγc, TSL-P-R, EB13, CLF-1, CNTF-Rα, gp130, Leptin-R, PRL-R, GH-R, Epo-R, Tpo-R, IFN-λR1, IFN-λR2, IFNR1 and IFNR2.

Furthermore, the immunomodulator is IL-10.

Furthermore, the amino acid sequence of the IL-10 is shown by SEQ ID NO:7, or is an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO:7.

Furthermore, the amino acid sequence of the second heavy chain is shown by SEQ ID NO:3, or is an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO:3.

Furthermore, the nucleotide sequence encoding the second heavy chain is shown by SEQ ID NO:6, or is a nucleotide sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO:6.

The present invention provides a method for the preparation of the above-mentioned heterodimeric protein by transferring three recombinant plasmids containing the light chain, the first heavy chain and the second heavy chain above-mentioned respectively into the same host cell for recombinant expression.

Further, the concentration ratio of the recombinant plasmids of the light chain, the first heavy chain and the second heavy chain is 1:0.5-2:0.5-2.

Furthermore, the concentration ratio of the recombinant plasmids of the light chain, the first heavy chain and the second heavy chain is 1:1:1.

Further, the host cell is a mammalian cell, bacteria, fungi or insect cell.

Furthermore, the mammalian cell is a CHO cell, SP20 cell, NSO cell, COS cell, BHK cell, HEK293 cell or PerC6 cell.

Furthermore, the mammalian cell is a CHO cell.

The present invention provides a nucleic acid encoding the heterodimeric protein as described above.

The present invention provides a vector or plasmid containing the nucleic acid as described above.

The present invention provides a cell expressing the vector or plasmid as described above.

The present invention also provides a pharmaceutical composition comprising the heterodimeric protein as described above and at least one pharmaceutically acceptable excipient, diluent or carrier.

Further, the pharmaceutical composition can be used alone or combined with other therapeutic agents to improve efficacy or reduce potential adverse effect.

The present invention also provides use of the heterodimeric protein as described above in the preparation of a drug for prevention and treatment of an oncological disease.

Further, the oncological disease comprises one or more of colorectal cancer, membranous adenocarcinoma, lung cancer, esophageal cancer, prostate cancer, pro-connective tissue proliferative small round cell tumor, ovarian cancer, gastric cancer, pancreatic cancer, liver cancer, kidney cancer, breast cancer, bon-small cell lung cancer, melanoma, alveolar rhabdomyosarcoma, embryonal rhabdomyosarcoma, Ewing's sarcoma, nephroblastoma, neuroblastoma, ganglioneuroma, medulloblastoma, high grade glioma, diffuse intrinsic pontine glioma, and embryonal tumor with multilayered rosettes.

The present invention also provides use of the heterodimeric protein as described above in the preparation of a reagent or kit for the detection of B7H3 and/or IL-10 receptor molecules.

The term “heterodimer” as used herein generally refers to a molecule (e.g. a proteinaceous molecule) composed of two different members. The two members of a heterodimer may differ in structure, function, activity and/or composition. For example, the two different members may comprise polypeptides differing in the order, number, or kind of amino acid residues forming these polypeptides. Each of the two different members of a heterodimer may independently comprise one, two or more units, polypeptide chains, or moieties.

The term “targeting moiety” as used herein generally refers to a molecule, complex or aggregate, that binds specifically, selectively or preferentially to a target molecule, cell, particle, tissue or aggregate. For example, a targeting moiety may be an antibody, antigen-binding antibody fragment, bispecific antibody or other antibody-based molecule or compound. Other examples of targeting moieties may include, but are not limited to, aptamers, avimers, receptor-binding ligands, nucleic acids, biotin-avidin binding pairs, binding peptides or proteins, etc. The terms “targeting moiety” and “binding moiety” are used interchangeably herein.

The term “antigen binding site” or “binding portion” as used herein generally refers to a part of an antibody that participates in antigen binding. An antigen binding site may be formed by amino acid residues of the N-terminal variable (“V”) regions of a heavy (“H”) chain and/or a light (“L”) chain. Three highly divergent stretches within the V regions of the heavy and light chains are referred to as “hypervariable regions” which are interposed between more conserved flanking stretches known as “framework regions” or “FRs”. In the antibody molecule, the three hypervariable regions of the light chain and the heavy chain are arranged opposite each other in three dimensions to form an antigen-binding ‘surface’. This surface mediates the recognition and binding of the target antigen.

There are multiple methods/systems in the field for defining and describing CDRs that have been developed and refined over the years, including Kabat, Chothia, IMGT, AbM and Contact. Kabat is the most commonly used and defines CDRs based on sequence variability; Chothia defines CDRs based on sequence variability based on the position of the structural loop region; IMGT system defines CDRs based on sequence variability and position within the variable domain structure; AbM is based on Oxford Molecular's AbM antibody modelling software and is a compromise between Kabat and Chothia; Contact defines CDRs based on the analysis of complex crystal structures and is similar to Chothia in several respects. Numbering of amino acid positions (e.g. amino acid residues in the Fc region) and target regions (e.g. CDR) in the present invention use Kabat system.

The term “tumor antigen” as used herein generally refers to an antigenic substance produced in or by tumor cells, which may have an ability to trigger an immune response in a host. For example, a tumor antigen may be a protein, a polypeptide, a peptide, or a fragment thereof, which constitutes part of a tumor cell and is capable of inducing tumor-specific cytotoxic T lymphocytes. In some embodiments, the term “tumor antigen” may also refer to biomolecules (e.g., proteins, carbohydrates, glycoproteins, etc.) that are exclusively or preferentially or differentially expressed on a cancer cell and/or are found in association with a cancer cell and thereby provide targets preferential or specific to the cancer. For example, the preferential expression can be preferential expression as compared to any other cell in the organism, or preferential expression within a particular area of the organism (e.g. within a particular organ or tissue).

The term “immune checkpoint” as used herein generally refers to suppressor and activator molecules in the immune system that regulate the body's anti-tumor immune system through modulation of T-cell activity. For example, the suppressor molecules include PDL1, B7H3, CTLA4, etc. and the activator molecules include OX40, 4-1BB, CD40, etc.

The term “immunomodulator” as used herein generally refers to substances that affect the function of the immune system. Immunomodulators may enhance or reduce the immune response. For example, the immunomodulator can be an active agent in immunotherapy, including but not limited to, a recombinant, synthetic and/or natural preparation of cytokines, granulocyte colony-stimulating factor (G-CSF), interferon, imiquimod, bacterial cell membrane fragments, chemokines, interleukins, cytosine phosphate-guanosine (CpG) oligodeoxynucleotides and dextran. In some embodiments, the immunomodulator is a cytokine.

The term “covalent bond” as used herein generally refers to a chemical bond formed between atoms by the sharing electrons. For example, a covalent bond may be polar or non-polar. In some embodiments, the covalent bond is a disulfide bond.

The term “polypeptide linker” as used herein generally refers to a synthetic amino acid sequence that links or couples two peptide sequences (e.g. links two peptide structural domains). A polypeptide linker may link two amino acid sequences through a peptide bond. In some embodiments, the polypeptide linker of the present application links an immunomodulator to the Fc region.

The term “antibody” as used herein generally refers to a protein comprising one or more polypeptides substantially encoded by an immunoglobulin gene or fragment of an immunoglobulin gene. Immunoglobulin genes may include κ, λ, α, γ, δ, ε and μ constant region genes and a variety of immunoglobulin variable region genes. As used herein, the light chain may be classified as κ or λ. The heavy chain may be classified as γ, μ, α, δ or ε, which in turn define the immunoglobulin classes as: IgG, IgM, IgA, IgD and IgE, respectively. Antibodies as used herein may have structural units comprising tetramers. Each tetramer may comprise two pairs of identical polypeptide chains, each pair having a “light” chain (approximately 25 kD) and a “heavy” chain (approximately 50-70 kD). The N-terminus of each member may define a variable region of approximately 100 to 110 or more amino acids which is primarily responsible for antigen recognition. As used herein, the terms variable light chain (VL) and variable heavy chain (VH) usually refer to these regions of the light and heavy chains, respectively. Antibodies may exist as intact immunoglobulins or as a variety of fully characterised fragments produced by digestion with various polypeptidases or by de novo expression.

The term “antibody” as used herein may also include antibody fragments produced by modification of the whole antibody or by de novo synthesis using recombinant DNA methods, including but not limited to Fab′2, IgG, IgM, IgA, IgE, scFv, dAb, nanobodies, single and double chain antibodies. In some embodiments, the antibodies comprise, but are not limited to, Fab′2, IgG, IgM, IgA, IgE and single chain antibodies, such as single chain Fv (scFv) antibodies, wherein the variable heavy chain and variable light chain are linked together (either directly or via peptide junctions) to form a continuous polypeptide.

In some embodiments, the antibodies and fragments of the present application are bispecific. In some embodiments, the bispecific antibody or fragment thereof has binding specificity for at least two different epitopes (e.g., at least one of the at least two different epitopes is a tumor-associated antigen). In some embodiments, the antibodies and fragments may also be heterologous antibodies, e.g. they may be or may comprise two or more linked antibodies or antibody-binding fragments (e.g. Fab), wherein each antibody or fragment has a different specificity.

The term “homology” as used herein generally refers to sequence similarity or exchangeability between two or more polynucleotide sequences or between two or more polypeptide sequences. In some embodiments, homologous polynucleotides are those sequences that are hybridized under stringent conditions and have at least 80% (e.g. 80%, 81%, 82%, 83%, 84%, 85%, 86%, 87%, 88%, 89%, 90%, 91%, 92%, 93%, 94%, 95%, 96%, 97%, 98%, 99% or 100%) of sequence identity.

The term “host cell” as used herein generally includes an individual cell, a cell line or cell culture which can be or has been a recipient for the subject plasmids or vectors, comprise the polynucleotide of the present disclosure, or express the heterodimeric protein (e.g. heterodimer protein) of the present disclosure. Host cells may include progeny of a single host cell. The progeny may not necessarily be completely identical (in morphology or in genomic of total DNA complement) to the original parent cell due to natural, accidental, or deliberate mutation. A host cell may include cells transfected in vitro with a vector of the present disclosure. A host cell may be a bacterial cell (e.g., E. coli), a yeast cell or other eukaryotic cells, e.g., a COS cell, a Chinese hamster ovary (CHO) cell, a HeLa cell, or a myeloma cell.

The term “vector” as used herein generally refers to a nucleic acid molecule capable of self-replication in a suitable host, which transfers the inserted nucleic acid molecule into and/or between host cells. The term may include vectors primarily for insertion of DNA or RNA into a cell, vectors primarily for replication of DNA or RNA, and expression vectors for transcription and/or translation of DNA or RNA. It also comprises vectors that perform more than one of these functions. An “expression vector” is a polynucleotide that can be transcribed and translated into a polypeptide when introduced into a suitable host cell.

The terms “treatment”, “cure”, “prevention” or “improvement” are used interchangeably herein and refer to a method for achieving beneficial or desired results including, but not limited to therapeutic benefit and/or preventive benefit. As used herein, therapeutic benefit generally refers to the elimination or reduction in severity of the underlying condition being treated. In addition, therapeutic benefit is achieved by eliminating, reducing the severity or the incidence of one or more physical symptoms associated with the underlying condition such that an improvement is observed in the subject (although the subject may still be afflicted with the underlying condition). For preventive benefit, the composition may be administered to subjects at risk of developing a specific disease, or who report one or more physical symptoms of the disease, even though a diagnosis of the disease may not have been made.

The term “reagent” as used herein generally refers to a biological part, pharmaceutical part or compound or other part. Non-limiting examples include simple or complex organic or inorganic molecules, peptides, proteins, oligonucleotides, antibodies, antibody derivatives, antibody fragments, vitamin derivatives, carbohydrates, toxins or chemotherapeutic compounds. Various compounds can be synthesized, such as small molecules and oligomers (e.g. oligopeptides and oligonucleotides) as well as synthetic organic compounds based on different core structures. In addition, various natural sources such as plant or animal extracts can provide compounds for screening.

The terms “anticancer agent”, “antineoplastic agent” or “chemotherapeutic agent” as used herein generally refers to any agent that can be used to treat a tumor condition. A class of anti-cancer agents comprises chemotherapeutic agents.

The term “chemotherapy” as used herein generally refers to the administration of one or more chemotherapeutic drugs and/or other agents to a cancer patient by various methods, including intravenous, oral, intramuscular, intraperitoneal, intravesical, subcutaneous, transdermal, oral or inhaled or in the form of suppositories.

The term “in vivo” as used herein generally refers to an event that takes place in a subject's body.

The term “in vitro” as used herein generally refers to an event that takes places outside of a subject's body. For example, an in vitro assay encompasses any assay conducted outside of a subject. In vitro assays encompass cell-based assays in which dead or living cells are employed. In vitro assays also encompass a cell-free assay in which no intact cells are employed.

The term “subject” as used herein generally refers to human or non-human animals, including but not limited to cats, dogs, horses, pigs, cattle, sheep, goats, rabbits, mice, rats or monkeys.

The term “room temperature” refers to 15-30° C.

### BRIEF DESCRIPTION OF THE DRAWINGS

FIG. 1 shows a schematic diagram of the constructed antibodies (ie. heterodimeric proteins) in the present invention.

FIG. 2 shows ELISA assay for the binding activity of the constructed antibodies to B7H3 protein.

FIG. 3 shows ELISA assay for the binding activity of the constructed antibodies to IL-10 receptor proteins.

FIG. 4 shows enhancement of the biological activity of CD8+ T cells by the constructed antibodies.

FIG. 5 shows anti-tumor biological activity of the constructed antibodies.

FIG. 6 shows anti-tumor biological activity of the constructed antibodies.

### DETAILED DESCRIPTION

The invention is further described below in connection with embodiments which are used to describe some specific embodiments of the invention and are not intended to limit the scope of protection of the invention.

Unless otherwise defined, all technical and scientific terms used herein have the same meaning as those generally understood by ordinary technicians in the field to which this application belongs. Although methods and materials similar to or equivalent to the methods and materials described herein may be used in the practice or testing of this application, appropriate methods and materials are described below. In case of contradiction, the patent specification shall prevail.

### Example 1 Acquisition and Optimization of Nucleotide Sequences

The light chain and heavy chain amino acid sequence information of the antibody was derived from the disclosed sequence information of the B7H3 target monoclonal antibody, and the variable and constant region information of the sequence was obtained by analysis (The amino acid sequence of the CH1-hinge-CH2-CH3 constant region of the IgG1 heavy chain is shown by SEQ ID NO: 8; the amino acid sequence of the CK constant region of the IgG1 light chain is shown by SEQ ID NO: 9; the amino acid sequence of the heavy chain of the B7H3 antibody is shown by SEQ ID NO: 10; the nucleotide sequence encoding the heavy chain of the B7H3 antibody is shown by SEQ ID NO: 11; the amino acid sequence of the variable region of the heavy chain of the B7H3 antibody is shown by SEQ ID NO: 12; and the amino acid sequence of the variable region of the light chain of the B7H3 antibody is shown by SEQ ID NO: 13). The natural IL-10 variant sequence (SEQ ID NO:7) is inserted into the amino acid sequence of a heavy chain. The Fc of the amino acid sequence of the antibody is adapted to other IgG types, such as IgG4, as required, and further amino acid mutations of the desired form are engineered into each heavy chain, resulting in a target antibody (heterodimeric protein) having an amino acid sequence of:

The first heavy chain is shown by SEQ ID NO: 1, the light chain is shown by SEQ ID NO: 2, and the second heavy chain is shown by SEQ ID NO: 3.

Each of the above target amino acid sequences was translated into nucleotide sequences, and a series of parameters that may affect antibody expression in mammalian cells: codon preference, GC content (i.e. the ratio of guanine G to cytosine C of the four bases of DNA), CpG islands (i.e. regions of the genome with a high density of CpG dinucleotides), secondary structure of mRNA, splice sites, pre-mature PolyA sites, internal Chi sites (a short segment of DNA in the genome near which homologous recombination has an increased chance of occurring) or ribosomal binding sites, RNA instability sequences, inverted repeat sequences and restriction enzyme cleavage sites that may interfere with cloning were optimized; relevant sequences that may improve translation efficiency, such as Kozak sequence, SD sequence, and termination codon were also added. A heavy chain gene and a light chain gene were designed to encode the above antibodies, respectively, and a nucleotide sequence encoding the signal peptide was designed at the 5′ end of the heavy chain and the light chain, respectively, optimized according to the amino acid sequence; in addition, a termination codon was added to the 3′ end of the light chain and heavy chain nucleotide sequences, respectively.

Finally, the optimized nucleotide sequence encoding the antibody was obtained as follows:

The nucleotide sequence encoding the first heavy chain is shown by SEQ ID NO: 4, the nucleotide sequence encoding the light chain is shown by SEQ ID NO: 5, and the nucleotide sequence encoding the second heavy chain is shown by SEQ ID NO: 6.

### Example 2 Gene Synthesis and Construction of Expression Vectors

The pcDNA3.1-G418 vector was used as a dedicated vector for the expression of the light and heavy chains of the multifunctional antibodies described. The pcDNA3.1-G418 vector comprises the CMV Promoter used for the heavy chain, the eukaryotic screening marker G418 tag and the prokaryotic screening tag Ampicilline. The nucleotide sequences (i.e. target genes) of the antibody-expressing the first heavy chain, the second heavy chain and light chain coding genes were obtained separately by gene synthesis, the vector and target fragments were double digested with HindIII and XhoI, recovered and then enzymatically ligated by DNA ligase and transformed into E. coli competent cell DH5a. Positive clones were selected and validated by plasmid extraction and enzymatic digestion to obtain recombinant plasmids containing the antibody the first heavy chain, the second heavy chain and light chain coding genes described.

### Example 3 Plasmid Extraction

The recombinant plasmids containing each of the above-mentioned target genes were transformed into E. coli competent cell DH5a, and the transformed bacteria were coated on LB plates containing 100 μg/mL ampicillin for incubation, and the plasmid clones were selected into liquid LB medium for incubation, and shaken at 260 rpm for 14 hours. The plasmids were extracted by the endotoxin-free plasmid large extraction kit, and dissolved in sterile water, and the concentration was determined with a nucleic acid protein quantifier.

### Example 4 Plasmid Transfection, Transient Expression and Antibody Purification

ExpiCHO was cultured to a cell density of 6×10⁶ cells/mL at 37° C., 8% CO₂, and 100 rpm. The constructed plasmids were transfected into the above cells by liposomes according to combination pairs. The concentration of the transfected plasmids was 1 mg/mL, and the volume of the liposomes was determined by reference to the ExpiCHO™ Expression System kit, and cultured at 32° C., 5% CO₂, and 100 rpm for 7-10 days. Feeding was given once after 18-22 h of transfection and once between day 5, respectively. The above culture product was centrifuged at 4000 g, and filtered through a 0.22 μm filter membrane and the supernatant of the medium was collected. The antibody proteins obtained were purified by Protein A and ionic column, and the eluent was collected.

The specific operation steps for Protein A and ionic column purification were as follows: cell culture fluid was centrifuged at high speed and the supernatant was taken, and affinity chromatography was performed using GE's Protein A chromatography column. The equilibrium buffer used for chromatography was 1×PBS (pH 7.4). After the cell supernatant was loaded and combined, it was washed with PBS until the ultraviolet rays returned to the baseline, and then the target protein was eluted with the elution buffer 0.1 M glycine (pH 3.0), and then the pH was adjusted to neutral using Tris for storage. The product from affinity chromatography was adjusted to pH of 1-2 pH units below or above pI, and appropriately diluted to control the sample conductance below 5 ms/cm. Appropriate corresponding pH buffers such as phosphate buffer, acetate buffer and other conditions, and conventional ion exchange chromatography methods in the field such as anion exchange or cation exchange were used to carry out NaCl gradient elution under the corresponding pH conditions, and the collection tubes where the target proteins were located were selected and combined for storage according to SDS-PAGE. Then, the eluent obtained after purification was ultrafiltrated into the buffer.

### Example 5 Detection of the Affinity of Antibodies to B7H3 by ELISA

HuB7H3-his (purchased from ACROBiosystems) was diluted to 0.5 g/mL with the PBS buffer of pH 7.4 and coated into 96-well ELISA plates at 100 μL/well and allowed to stand overnight at 4° C. After the plate was blocked with 1% BSA blocking solution for 1 hour, the plate was washed three times with PBST and the purified antibody was diluted to 10 μg/mL with 0.5% BSA sample diluent as the starting concentration and 3-fold diluted in 11 gradients and set up an irrelevant antibody negative control and a B7H3 chimeric antibody positive control. (B7H3 chimeric antibody sequence source: Mahiuddin, Ahmed, Ming, et al. Humanized Affinity-matured Monoclonal Antibody 8H9 Has Potent anti-tumor Activity and Binds to FG Loop of Tumor antigen B7H3. [J]. The Journal of biological chemistry, 2015.), and it was added 100 μL per well, incubated for 1 h at 37° C. The plate was then washed 3 times with PBST and HRP-labelled goat anti-human IgGFc was diluted according to 1:20,000 with sample diluent and it was added 100 μL per well and incubated for 1 h at room temperature. After washing the plate 4 times with PBST, it was added 100 μL of TMB substrate per well and incubated at room temperature without light for 10 min. 100 μL of 1 M HCl was added to each well to terminate the colour development reaction. The absorbance value of each well in the 96-well plate was measured on a multifunctional enzyme standard at a wavelength of 450 nm and a reference wavelength of 570 nm, absorbance value (OD) per well=OD450 nm-OD570 nm. The concentrations of the antibodies were logarithmically plotted as the horizontal coordinate and the measured absorbance values per well as the vertical coordinate. Sigmoidal dose-response (Variable Slope) method (Graph Pad Prism Software, Graph Pad Software, SanDiego, California) was chosen for non-linear regression to obtain the binding curve of the target antibody to B7H3 protein.

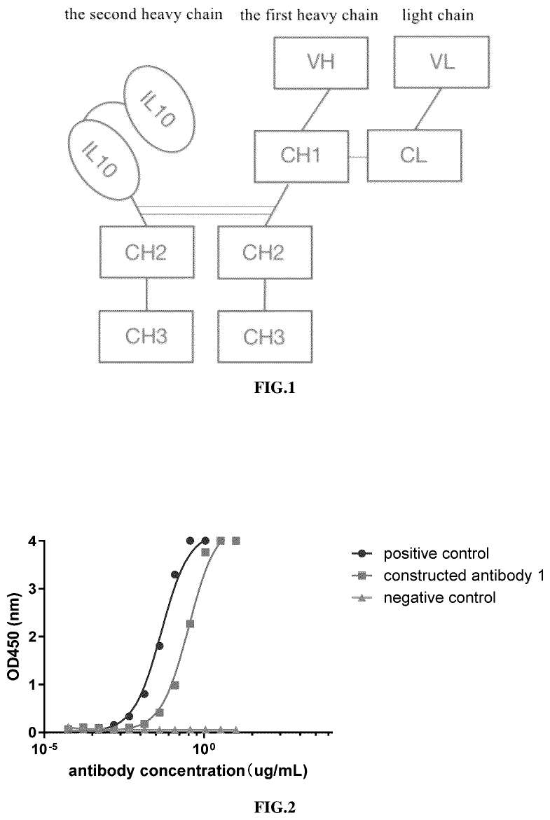

The ELISA results of the constructed antibodies are shown in FIG. 2, and the constructed antibodies can bind to B7H3 in multiple concentration ranges.

### Example 6 Detection of the Affinity of the Antibodies to IL-10 Receptor by ELISA

The IL-10 receptor human IL10RA-his (purchased from Beijing Yiqiao Shenzhou Technology Co., Ltd.) was diluted to 0.5 μg/mL with the PBS buffer of pH 7.4 and coated into 96-well ELISA plates at 100 μL/well and allowed to stand overnight at 4° C. The plate was blocked with 1% BSA blocking solution for 1 hour, and then washed 3 times with PBST. The purified antibody was diluted to 10 μg/mL with 0.5% BSA sample diluent as the starting concentration and 3-fold diluted in 11 gradients, and set up a negative control with irrelevant antibodies and a positive control (IL-10), and it was added 100 μL per well, incubated for 1 h at 37° C. The plate was washed 3 times with PBST and HRP-labelled goat anti-human IgG Fc (Jackson Cat: 109-035-098) was diluted according to 1:10000 with sample diluent and it was added 100 μL per well and incubated for 1 h at room temperature. After washing the plate 4 times with PBST, it was added 100 μL of TMB substrate per well and incubated at room temperature without light for 10 min. 100 μL of 1 M HCl was added to each well to terminate the colour development reaction.

The absorbance value of each well in the 96-well plate was measured on a multifunctional enzyme standard at a wavelength of 450 nm and a reference wavelength of 570 nm, absorbance value (OD) per well=OD450 nm-OD570 nm. The concentration of the antibodies were logarithmically plotted as the horizontal coordinate and the measured absorbance value per well as the vertical coordinate. Sigmoidal dose-response (Variable Slope) method (Graph Pad Prism Software, Graph Pad Software, SanDiego, California) was chosen for non-linear regression to obtain the binding curve of the target antibody to the IL-10 receptor IL10RA protein.

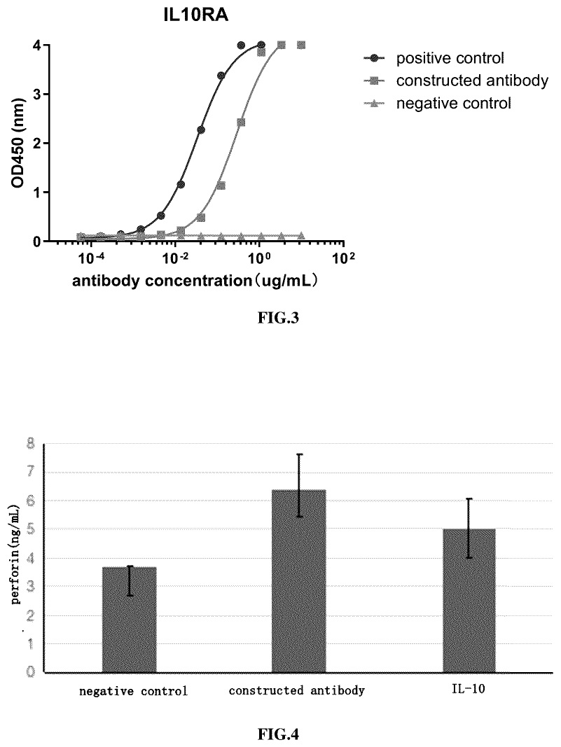

The ELISA results of the constructed antibodies are shown in FIG. 3 respectively, and the constructed antibodies can bind to the IL-10 receptor in multiple concentration ranges.

### Example 7 IL-10 Biological Activity of the Constructed Antibodies

When the constructed antibody is co-incubated with CD8+ T cells, its IL-10 end will bind to the IL-10 receptor on the surface of CD8+ T cells. The effect of the constructed antibody in promoting the secretion of perforin by CD8+ T cells was tested to verify whether the constructed antibody enhanced the cytotoxicity of CD8+ T cells. A working concentration at 10 μg/mL of CD3 and 2 μg/mL of CD28 protein was used to coat a 6-well plate at 4° C. overnight to allow sufficient binding of the protein to the plate, and 3 wells were set up at 2 mL/well. The next day the coating solution was discarded and washed three times with PBS. Fresh CD8+ T cells were purchased from Shanghai Auxeres Biotechnology Co., Ltd. and the CD8+ T cells were centrifuged (400 g, 10 min) according to the fresh cell processing protocol. Cells were resuspended in 5 mL 1640 complete medium and counted. Cell density was adjusted to 2.5×10⁶ cells/mL with 1640 complete medium, 2 mL/well was added to CD3 and CD28 protein-coated plates, mixed well and co-stimulated for 70-72 h in a 37° C. incubator.

All CD8+ T cells were collected from 6-well plates, centrifuged (400 g, 10 min), resuspended with 5 mL of 1640 complete medium and counted, and the cell density was adjusted to 1.6×10⁶ cells/mL with 1640 complete medium, and was added to 24-well plates at 250 μL/well and set aside. The constructed antibody, negative control (B7H3 monoclonal antibody), and IL-10 (STEMCELL, Cat: 78036) were first diluted to 20 nM in 1640 complete medium and then 10-fold diluted in 4 concentration gradients and 2 replicate wells. After the dilution is completed, it is added to the corresponding concentration setting wells at 250 μL/well. Blank control wells were supplemented with sample dilution at 250 μL/well, mixed well, and co-stimulated for 70-72 h at 37° C. in an incubator.

Cells were treated with samples for 3 days, and cells from all sample groups were counted respectively and the cell number of the group with the least cell number was used as a standard, and the same number of cell was taken from each well respectively and centrifuged (400 g, 10 min). The 1640 medium containing 1 μg/mL soluble CD3 protein was used to resuspend the cells. The cells were spread 500 μL/well in a 24-well plate and mixed. The supernatants of each group were collected after incubation at 37° C. for 4 h. The commercial perforin cytokine assay kit was used to detect the secretion of stimulated perforin by the constructed antibodies on CD8+ T cells and the results of the assay are shown in FIG. 4. IL-10 significantly stimulated perforin secretion from CD8+ T cells in a concentration-dependent manner. the constructed antibodies at high concentration significantly stimulated perforin secretion from CD8+ T cells, while the B7H3 antibody failed to stimulate perforin secretion from CD8+ T cells, indicating that the stimulation of perforin secretion from CD8+ T cells by the constructed antibodies are dependent on the end of IL-10.

### Example 8 In Vivo Anti-Tumor Activity of the Constructed Antibodies

A xenograft tumor model was constructed by subcutaneously injecting 5×10⁶ B7H3-expressing human gastric cancer cell line Hs-746T cells into the right dorsum of female nude mice, and group administration of drugs was initiated when the average tumor volume reached 100 mm³. 10 mpk constructed antibody, 10 mpk isotype control, or an equal volume of PBS was used for the treatment of intraperitoneal injections, which were administered every 3 days and twice a week. The experimental index was to examine whether tumor growth was inhibited, retarded or cured. Tumor diameter was measured three times a week. The tumor volume was calculated as V=0.5a×b², where a and b represent the long and short diameters of the tumor, respectively.

The results are shown in FIG. 5, where the horizontal coordinate indicates the number of days after inoculation with Hs-746T cells and the vertical coordinate indicates the tumor volume. 6 days after the starting cell inoculation, 100 mm³ was reached for cage splitting and drug administration, and 21 days after drug administration, the average tumor volume of mice in the PBS control and isotype control treatment groups reached 2343±367 mm³; while the tumor volume of mice in the constructed antibody treatment group was only 40.6±40.6 mm³, with significant inhibition of tumor growth and tumor regression in some of mice in the constructed antibody treatment group. The constructed antibody shows good anti-tumor activity.

### Example 9 In Vivo Anti-Tumor Activity of the Constructed Antibodies

A subcutaneous graft tumor model was established by subcutaneously injecting 5×10⁶ human B7H3-expressing murine colorectal cancer cell line hB7H3-MC38 cells into the right back of female nude mice, and group administration of drugs was initiated when the average tumor volume reached 80 mm³ or 200 mm³. It was divided into 5 groups: (1) G1: PBS group; (2) G2: 0.3 mpk group; (3) G3: 1 mpk group; (4) G4: 3 mpk group; and (5) G5: 3 mpk group. Five groups of mice were treated by intraperitoneal injection, which was administered twice a week for eight doses. The experimental index was to examine whether the tumor growth was inhibited, retarded or cured. The tumor diameter was measured three times a week. The calculation formula for tumor volume is: V=0.5a×b², where a and b represent the long and short diameters of the tumor, respectively.

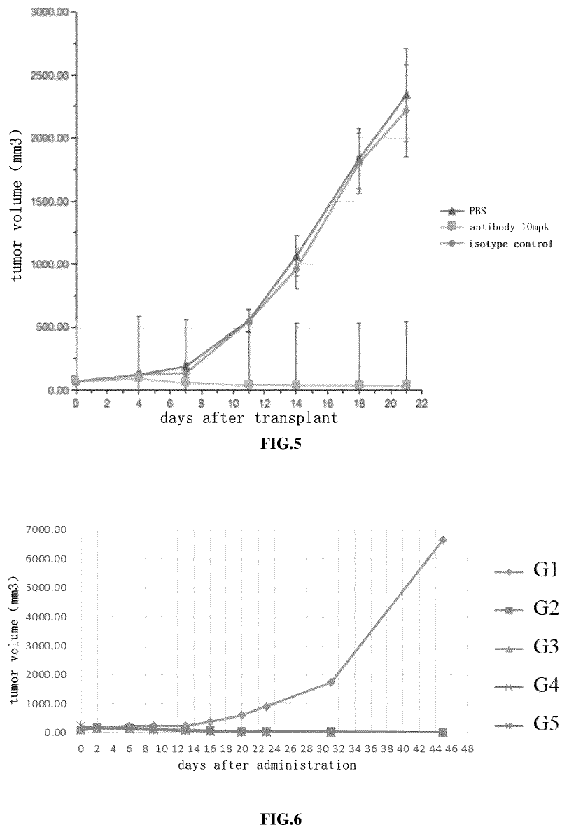

The results are shown in FIG. 6, where the horizontal coordinate indicates the number of days after group administration and the vertical coordinate indicates the tumor volume. 4 days after the initiation of cell inoculation, 80 mm³ was reached for cage splitting, and G1, G2, G3 and G4 groups were treated by intraperitoneal injection. 6 days after the initiation of cell inoculation, 200 mm³ was reached for cage splitting, and G5 group was treated by intraperitoneal injection. After 31 days of administration, the average tumor volume of mice in G1 group (PBS control group) reached 1708.63±602.05 mm³, while the tumor volume of mice in the constructed antibody treatment group was completely regressed except for G2 group (0.3 mpk administration group), and the tumor volume of G2 group (0.3 mpk administration group) was only 10.84±6.86 mm³. The constructed antibody shows good anti-tumor activity.

It should be noted that the above is only a preferred embodiment of the present invention and is not intended to limit the scope of the present invention. Any modifications, equivalent replacements and improvements etc. made within the spirit and principles of the present invention shall be included within the scope of protection of the present invention.

1. A heterodimeric protein, comprising:
(1) a light chain and a first heavy chain, wherein the light chain and the first heavy chain are combined to form a targeting moiety that exhibits binding specificity to a tumor antigen or an immune checkpoint; and
(2) a second heavy chain, wherein the second heavy chain comprises a Fc region and an immunomodulator fused to the Fc region;
wherein the light chain, the first heavy chain, and the second heavy chain are combined to form the heterodimeric protein.

2. The heterodimeric protein of claim 1, wherein the tumor antigen or the immune checkpoint is selected from one or more of the group consisting of: B7H3, B7H4, B7H5, BTLA, CD27, CD28, CD153, CD40, CD40L, CD70, CD80, CD86, CD96, CD112, CD134, CD137, CD137L, CD152/CTLA-4, CD155, CD223, CD226, CD252/OX40L, CD258, CD273/PD-L2, CD274/PD-L1, CD278, CD279, CD357, DR3, Galectin-9, GITRL, HVEM, ICOSL/B7RP1/B7H2, IDO, TIGIT, TIM-3, TLIA, MART-1/MelanA, gp100, tyrosinase, TRP-1, TRP-2, MAGE-1, MAGE-3, BAGE, GAGE-1, GAGE-2, p15, CEA, p53, Ras, HER-2/neu, BCR-ABL, E2A-PRL, H4-RET, IGH-IGK, MYL-RAR, Epstein Barr virus antigen EBVA, human papillomavirus antigen E6 or E7, TSP-180, MAGE-4, MAGE-5, MAGE-6, RAGE, NY-ESO, erbB, p185erbB2, p180erbB-3, c-met, nm-23H1, PSA, TAG-72, CA 19-9, CA 72-4, CAM 17.1, NuMa, K-ras, β-linked protein, CDK4, Mum-1, p 15, p 16, 43-9F, 5T4, 791Tgp72, methotrexate, β-HCG, BCA225, BTAA, CA 125 CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, MUC16, IL13Rα2, FRα, VEGFR2, Lewis Y, FAP, EphA2, CEACAM5, EGFR, CA6, CA9, GPNMB, EGP1, FOLR1, endothelial receptor, STEAP1, SLC44A4, binding element-4, AGS-16, guanylyl cyclase C, MUC-1, CFC1B, integral protein α3 chain, TPS, CD19, CD20, CD22, CD30, CD72, CD180, CD171, CD123, CD133, CD138 CD37, CD70, CD79a, CD79b, CD56, CD74, CD166, CD71, CLL-1/CLEC12A, ROR1, phosphatidylinositol proteoglycan 3, mesothelin, CD33/IL3Ra, c-Met, PSCA, PSMA, glycolipid F77, EGFRVIII, BCMA, GD-2, MY-ESO-1 and MAGE A3.

3. The heterodimeric protein of any one of claims 1-2, wherein the immunomodulator is cytokine, cytokine receptor, growth factor, hormone or extracellular matrix molecule.

4. The heterodimeric protein of claim 1, wherein both the light chain and the first heavy chain contain a complementary determination region containing amino acid sequences having at least 80% identity to the corresponding CDR amino acid sequences of the light chain and the heavy chain of the antibody specifically binding to the tumor antigen or the immune checkpoint; the light chain of the antibody specifically binding to the tumor antigen or the immune checkpoint contains LCDR1 of the amino acid sequence shown by SEQ ID NO:17, LCDR2 of the amino acid sequence shown by SEQ ID NO:18, and LCDR3 of the amino acid sequence shown by SEQ ID NO: 19; and the first heavy chain of the antibody specifically binding to the tumor antigen or the immune checkpoint contains HCDR1 of the amino acid sequence shown by SEQ ID NO:14, HCDR2 of the amino acid sequence shown by SEQ ID NO:15, and HCDR3 of the amino acid sequence shown by SEQ ID NO:16.

5. The heterodimeric protein of claim 4, wherein the second heavy chain comprises two or more immunomodulators of the same or different types, and the two or more immunomodulators are fused to each other and to the Fc region; the immunomodulator is IL-10; and the second heavy chain has an amino acid sequence shown by SEQ ID NO:3 or is an amino acid sequence having at least 80% identity to SEQ ID NO:3.

6. (canceled)

7. (canceled)

8. (canceled)

9. A pharmaceutical composition comprising the heterodimeric protein of claim 1 and at least one pharmaceutically acceptable excipient, diluent, or carrier.

10. Use of the heterodimeric protein of claim 1, comprising:
(a) preparation of a drug for the treatment of an oncological disease comprising one or more of colorectal cancer, membrane adenocarcinoma, lung cancer, esophageal cancer, prostate cancer, pro-connective tissue proliferative small round cell tumor, ovarian cancer, gastric cancer, pancreatic cancer, liver cancer, kidney cancer, breast cancer, non-small cell lung cancer, melanoma, alveolar rhabdomyosarcoma, embryonal rhabdomyosarcoma, Ewing's sarcoma, nephroblastoma, neuroblastoma, ganglioneuroma, medulloblastoma, high-grade glioma, diffuse intrinsic pontine glioma, and embryonal tumor with multilayered rosettes; or (b) preparation of a reagent or a kit for the detection of B7H3 and/or IL-10 receptor molecules.
